(19) United States
(12) Patent Application Publication
(10) Pub. No.: US 2012/0142990 A1
(43) Pub. Date: Jun. 7, 2012

(54) PROCRESS FOR THE PREPARATION OF AN OLEFINIC PRODUCT, PROCESS FOR THE MANUFACTURE OF AN OXYGENATE CONVERSION CATALYST AND AN OXYGENATE CONVERSION CATALYST

(76) Inventors: Leslie Andrew Chewter, Amsterdam (NL); Jeroen Vsn Westrenen, Amsterdam (NL); Ferry Winter, Amsterdam (NL)

(21) Appl. No.: 13/320,973

(22) PCT Filed: Apr. 29, 2010
(86) PCT No.: PCT/EP2010/055844

§ 371 Date: Jan. 4, 2012

(30) Foreign Application Priority Data
May 19, 2009 (EP) 09160664.0

(51) Int. Cl. C07C 1/20; B01J 29/85; B01J 29/70; B01J 37/08

(52) U.S. Cl. 585/638; 502/60

(57) ABSTRACT

A process for the preparation of an olefinic product in the presence of an oxygenate conversion catalyst, the catalyst comprising a molecular sieve, wherein the molecular sieves comprises a SAPO-type silicoaluminophosphate, MFI-type aluminosilicate and/or MEL-type aluminosilicate, and a sulphur species at an elemental sulphur loading of at least 0.05 wt % based on total catalyst, the process comprising reacting an oxygenate feedstock under oxygenate conversion conditions to produce an olefinic reaction product. Further a process for the manufacture of an oxygenate conversion catalyst, and an oxygenate conversion catalyst comprising a molecular sieve having n-membered ring channels wherein n is 10 or less, and a sulphur species.

This invention relates to a process for the preparation of an olefinic product, a process for the manufacture of an oxygenate conversion catalyst and an oxygenate conversion catalyst.

Processes for the preparation of olefinic products from oxygenates are known in the art. Of particular interest is often the production of light olefins, in particular ethylene and/or propylene. The oxygenate feedstock can for example comprise methanol and/or dimethyl ether, and an interesting route includes their production from synthesis gas derived from e.g. natural gas or via coal gasification.

For example, WO2007/135052 discloses a process wherein an alcohol and/or ether containing oxygenate feedstock and an olefinic co-feed are reacted in the presence of a zeolite having one-dimensional 10-membered ring channels to prepare an olefinic reaction mixture, and wherein part of the obtained olefinic reaction mixture is recycled as olefinic co-feed. With a methanol and/or dimethyl ether containing feedstock, and an olefinic co-feed comprising C4 and/or C5 olefins, an olefinic product rich in light olefins can be obtained.

Various by-products are normally formed in the oxygenate to olefin reaction, such as aromatics and saturated hydrocarbons. In some cases this results in uneconomical streams being produced and for certain by-products the catalysts may be coked and deactivated. Thus the saturates make and aromatic make of an oxygenate to olefins reaction is preferably minimised.

There is a need for an improved and efficient oxygenate-to-olefins process wherein a minimum of by-products is formed.

According to a first aspect of the present invention there is provided a process for the preparation of an olefinic product in the presence of an oxygenate conversion catalyst, the catalyst comprising a molecular sieve, wherein the molecular sieves comprises a SAPO-type silicoaluminophosphate, MFI-type aluminosilicate, MEL-type aluminosilicate and/or comprises an aluminosilicate having one-dimensional 10-membered ring channels, and a sulphur species at an elemental sulphur loading of at least 0.05 wt % based on total catalyst, the process comprising reacting an oxygenate feedstock in oxygenate conversion conditions to produce an olefinic reaction product.

Preferably the sulphur species is an inorganic sulphur species, more preferably comprises an SO_{X} species such as at least one of SO₄²⁻ (sulphate) and SO₃ (sulfite). The catalyst may comprise from 0.05 to 2 wt % sulphur, preferably of from 0.1 wt % to 1 wt % based on total catalyst. The amount of sulphur is based on the elemental weight of sulphur (which does not need to be in elemental form though) and not on the total weight of sulphur species present. This may be determined by elemental analysis and is also referred to as elemental sulphur loading.

The catalyst may further comprise phosphorus or a phosphorus species. The phosphorus may be present in an amount of from 0.05 to 15 wt % based on total catalyst.

The expression ‘molecular sieve’ is used in the description and claims for a material containing small regular pores and/or channels and exhibiting catalytic activity in the conversion of oxygenate to olefin. Where reference is made in the description and in the claims to a molecular sieve, this can in particular be a zeolite. A zeolite is understood to be an aluminosilicate molecular sieve. The molecular sieve typically comprises molecular sieve having more-dimensional channels (“more-dimensional molecular sieve”). The molecular sieve can for example comprise a FER type, molecular sieve, MFI-type, MEL-type or a SAPO-type molecular sieve, or combinations thereof.

Preferably the molecular sieve having more-dimensional channels has a silica-to-alumina ratio (SAR) in the range from 1 to 1000. A SAR of 60 or higher is preferred, in particular 80 or higher, more preferably 100 or higher, still more preferably 150 or higher, such as 200 or higher. At higher SAR the percentage of C4 saturates in the C4 totals produced is minimized. The SAR is defined as the molar ratio of SiO₂/Al₂O₃ corresponding to the composition of the molecular sieve.

For certain embodiments, the molecular sieve comprises a first molecular sieve having more-dimensional channels and also comprises a further molecular sieve having only 10-membered ring channels in one direction which are not intersected by other channels, in particular other 8, 10 or 12-membered ring channels, from another direction (“one-dimensional molecular sieve”). The molecular sieve having one-dimensional 10-membered ring channels and/or the molecular sieve having more-dimensional channels can in particular be a zeolite or zeolites. The one-dimensional and/or the more-dimensional molecular sieve can be a mixture of different types of molecular sieves having the respective channel structures. So, for example, a mixture of ZSM-22 and ZSM-23 zeolites, both having one-dimensional 10-membered ring channels, can be used as one-dimensional molecular sieve. Similarly, different more-dimensional molecular sieves can be mixed to form the second molecular sieve.

Preferably, the further molecular sieve is selected from the group of TON-type (for example zeolite ZSM-22), MTT-type (for example zeolite ZSM-23), STF-type (for example SSZ-35), SFF-type (for example SSZ-44), EUO-type (for example ZSM-50), and EU-2-type molecular sieves or mixtures thereof.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type molecular sieves are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type molecular sieves are more particularly described in e.g. U.S. Pat. No. 4,397,827. For purposes of the present invention, EU-2 is considered to include its isotypes, e.g., ZSM-48.

In a further preferred embodiment a further molecular sieve of the MTT-type, such as ZSM-23, and/or a TON-type, such as ZSM-22 is used.

Molecular sieve and zeolite types are for example defined in Ch. Baerlocher and L. B. McCusker, Database of Zeolite Structures: http://www.iza-structure.org/databases/, which database was designed and implemented on behalf of the Structure Commission of the International Zeolite Association (IZA-SC), and based on the data of the 4th edition of the Atlas of Zeolite Structure Types (W. M. Meier, D. H. Olson and Ch. Baerlocher). The _i Atlas of Zeolite Framework Types, 5th revised edition 2001 and 6^{th} edition 2007 may also be consulted.

In special embodiments the oxygenate conversion catalyst can comprise less than 35 wt % of the first molecular sieve, based on the total molecular sieve in the oxygenate conversion catalyst, in particular less than 20 wt %, more in particular less than 18 wt %, still more in particular less than 15 wt %.

The weight ratio between the further molecular sieve having one-dimensional 10-membered ring channels, and the first molecular sieve having more-dimensional channels can be in the range of from 1:100 to 100:1, preferably in the range of from 1:1 to 100:1, more preferably in the range of 9:1 to 2:1.

In one embodiment molecular sieves in the hydrogen form are used in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. When the molecular sieve(s) are prepared in the presence of organic cations the molecular sieve may be activated by heating in an inert or oxidative atmosphere to remove organic cations, for example, by heating at a temperature over 500° C. for 1 hour or more. The zeolite is typically obtained in the sodium or potassium form. The hydrogen form can then be obtained by an ion exchange procedure with ammonium salts followed by another heat treatment, for example in an inert or oxidative atmosphere at a temperature over 300° C. The molecular sieves obtained after ion-exchange are also referred to as being in the ammonium form.

Preferably the further molecular sieve having one-dimensional 10-membered ring channels has a silica-to-alumina ratio (SAR) in the range of from 1 to 500. A particularly suitable SAR is less than 200, in particular 150 or less. A preferred range is from 10 to 200 or from 10-150.

For ZSM-22, a SAR in the range of 40-150 is preferred, in particular in the range of 70-120. Good performance in terms of activity and selectivity has been observed with a SAR of about 100.

For ZSM-23, an SAR in the range of 20-120 is preferred, in particular in the range of 30-80. Good performance in terms of activity and selectivity has been observed with a SAR of about 50.

In a special embodiment the reaction is performed in the presence of a more-dimensional molecular sieve, such as ZSM-5 or ZSM-11. Suitably to this end the oxygenate conversion catalyst comprises at least 1 wt %, based on total molecular sieve in the oxygenate conversion catalyst, of the first molecular sieve having more-dimensional channels, in particular at least 5 wt %, more in particular at least 8 wt %. The molecular sieve having more-dimensional channels is understood to have intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. The more-dimensional molecular sieve can be for example a FER type zeolite which is a two-dimensional structure and has 8- and 10-membered rings intersecting each other. Preferably however the intersecting channels in the more-dimensional molecular sieve are each 10-membered ring channels. Thus the more-dimensional molecular sieve may be a zeolite, or a SAPO-type (silicoaluminophosphate) molecular sieve. More preferably however the more-dimensional molecular sieve is a zeolite. A suitable more-dimensional molecular sieve is an MFI-type aluminosilicate, in particular zeolite ZSM-5. Another suitable more-dimensional molecular sieve is a MEL-type aluminosilicate, in particular zeolite ZSM-11.

The presence of the more-dimensional molecular sieve in the oxygenate conversion catalyst was found to improve stability (slower deactivation during extended runs) and hydrothermal stability compared to a catalyst with only the one-dimensional molecular sieve and without the more-dimensional molecular sieve. Without wishing to be bound by a particular hypothesis or theory, it is presently believed that this is due to the possibility for converting larger molecules by the molecular sieve having more-dimensional channels, that were produced by the molecular sieve having one-dimensional 10-membered ring channels, and which would otherwise form coke. When the one-dimensional aluminosilicate and the more-dimensional molecular sieve are formulated such that they are present in the same catalyst particle, such as in a spray-dried particle, this intimate mix was found to improve the selectivity towards ethylene and propylene, more in particular towards ethylene. The weight ratio between the molecular sieve having one-dimensional 10-membered ring channels, and the further molecular sieve having more-dimensional channels can be in the range of from 1:100 to 100:1. Preferably the further molecular sieve is the minority component, i.e. the above weight ratio is 1:1 to 100:1, more preferably in the range of 9:1 to 2:1.

In one embodiment the oxygenate conversion catalyst can comprise more than 50 wt %, preferably at least 65 wt %, based on total molecular sieve in the oxygenate conversion catalyst, of the molecular sieve having one-dimensional 10-membered ring channels. The presence of a majority of such molecular sieve strongly determines the predominant reaction pathway.

Without wishing to be bound by a particular hypothesis or theory, it is considered that the outer surface of the molecular sieve(s) and surface at the entrance of the channels has poor selectivity to the intended olefinic product whereas channels in the molecular sieve(s) have better selectivity towards the intended olefinic product, and it is considered that the sulphur treatment according to the present invention preferentially inhibits the activity of the outer surface of the molecular sieve(s) compared to the channels in the molecular sieves. Acid sites on the outer surface and acid sites at the entrance of the channels of the molecular sieve are thought to be a cause of unwanted by-product formation. This is particularly pronounced for molecular sieves present as relatively small crystals therefore having a relatively large surface-to-volume ratio. In a catalyst formulation including a molecular sieve with one-dimensional 10-membered ring channels and a more-dimensional molecular sieve it is believed that there are more unwanted by-product reactions caused by the more-dimensional molecular sieve. One factor can be that the more-dimensional molecular sieve is typically present as similar or smaller crystals than the one-dimensional molecular sieve, i.e. has a higher surface-to-volume ratio. It is presently believed that the present invention particularly passivates outer surface acid sites and the acid sites at the entrance of the channels on the more-dimensional molecular sieve component, such as of MFI-type, in particular ZSM-5.

Preferably the catalyst has particles comprising the first molecular sieve having more-dimensional channels, and the further molecular sieve having one-dimensional 10-membered ring channels, such that the individual catalyst particles comprise both the first molecular sieve and the further molecular sieve.

Thus typically the first and further molecular sieves are intimately mixed, that is crystals of the first and further molecular sieves are present in the same particle, rather than a mixture of formulated catalyst particles where individual particles have one or other of the molecular sieves, not both. Preferably therefore an average distance between a crystal of the first molecular sieve and a crystal of the further molecular sieve is less than an average particle size of the catalyst particles, preferably 40 μm or less, more preferably 20 μm or less, especially 10 μm or less. For near-spherical particles the average particle size can be determined by the weight-averaged diameter of a statistically representative quantity of particles, such as of e.g. 10 mg, 100 mg, 250 mg, or 1 g of particles. Such a statistically representative quantity of particles is referred to herein as a bed of particles. For other shapes of catalyst particles the skilled person knows how to define a suitable average of a characteristic dimension as average particle size, preferably a weight-average is used. The average distance between a crystal of the first molecular sieve and a crystal of the second molecular sieve can be determined using for instance electron-microscopy.

The catalyst may be prepared by spray drying and to provide an intimately mixed catalyst, the first and further molecular sieves can be mixed together before they are spray dried. Alternatively the first and further molecular sieves can be co-crystallised or intergrown in order to form the intimately mixed molecular sieves. For such embodiments a matrix can be added after co-crystallisation and the resulting mixture then spray dried. Co-crystallisation and intergrowth of two or more molecular sieves are well known processes to the skilled person and does not need any further explanation.

The molecular sieve(s) can be used as such or in a formulation, such as in a mixture or combination with a so-called binder material and/or a filler material, and optionally also with an active matrix component. Other components can also be present in the formulation. If one or more molecular sieves are used as such, in particular when no binder, filler, or active matrix material is used, the molecular sieve itself is/are referred to as oxygenate conversion catalyst. In a formulation, the molecular sieve(s) in combination with the other components of the mixture such as binder and/or filler material is/are referred to as oxygenate conversion catalyst.

It is desirable to provide a catalyst having good mechanical or crush strength, because in an industrial environment the catalyst is often subjected to rough handling, which tends to break down the catalyst into powder-like material. The latter causes problems in the processing. Preferably the molecular sieve is therefore incorporated in a binder material. Examples of suitable materials in a formulation include active and inactive materials and synthetic or naturally occurring zeolites as well as inorganic materials such as clays, silica, alumina, silica-alumina, titania, zirconia and aluminosilicate. For present purposes, inert materials, such as silica, are preferred because they may prevent unwanted side reactions which may take place in case a more acidic material, such as alumina or silica-alumina is used. The matrix material may be selected from the group consisting of: silica, magnesia, titania, kaolin, montmorillonite; preferably kaolin. Where kaolin is used, preferably it has less than 3 wt %, preferably less than 1.5 wt % iron, and preferably less than 4 wt %, preferably less than 3 wt % titania; all based on total content of kaolin.

The skilled artisan knows that silica binders can be prepared at low and high pH stabilized by alkaline (Na⁺), ammonium (NH₄⁺) and/or by acid (H⁺). A silica binder that is useful for obtaining spray dried catalyst with good attrition resistance, the binder is stabilized at very low pH (<1.5) or with high alkaline content. High alkaline is preferred, since low pH stabilization may influence the molecular sieve in such environment.

The oxygenate conversion catalyst particles preferably have an average particle size of less than 100 microns.

The oxygenate feedstock suitably comprises oxygenate species having an oxygen-bonded methyl group, such as methanol or dimethyl ether. Preferably the oxygenate feedstock comprises at least 50 wt % of methanol and/or dimethyl ether, more preferably at least 80 wt %, most preferably at least 90 wt %.

The oxygenate feedstock can be obtained from a different or separate reactor, which converts methanol at least partially into dimethyl ether. In this way, water may be removed by distillation and so less water is present in the process of converting oxygenate to olefins, which has advantages for the process design and lowers the severity of hydrothermal conditions the catalyst is exposed to.

The oxygenate feedstock can comprise an amount of water, preferably less than 10 wt %, more preferably less than 5 wt %. Preferably the oxygenate feedstock contains essentially no hydrocarbons other than oxygenates, i.e. less than 5 wt %, preferably less than 1 wt %.

In one embodiment, the oxygenate is obtained as a reaction product of synthesis gas. Synthesis gas can for example be generated from fossil fuels, such as from natural gas or oil, or from the gasification of coal. Suitable processes for this purpose are for example discussed in Industrial Organic Chemistry, Klaus Weissermehl and Hans-Jürgen Arpe, 3rd edition, Wiley, 1997, pages 13-28. This book also describes the manufacture of methanol from synthesis gas on pages 28-30.

In another embodiment the oxygenate is obtained from biomaterials, such as through fermentation. For example by a process as described in DE-A-10043644.

Preferably the oxygenate feedstock is reacted to produce the olefinic product in the presence of an olefinic co-feed. By an olefinic composition or stream, such as an olefinic product, product fraction, fraction, effluent, reaction effluent or the like is understood a composition or stream comprising one or more olefins, unless specifically indicated otherwise. Other species can be present as well. Apart from olefins, the olefinic co-feed may contain other hydrocarbon compounds, such as for example paraffinic compounds. Preferably the olefinic co-feed comprises an olefinic portion of more than 50 wt %, more preferably more than 60 wt %, for example more than 70 wt %, which olefinic portion consists of olefin(s). The olefinic co-feed can also consist essentially of olefin(s).

Any non-olefinic compounds in the olefinic co-feed are preferably paraffinic compounds. Such paraffinic compounds are preferably present in an amount in the range of from 0 to 50 wt %, more preferably in the range of from 0 to 40 wt %, still more preferably in the range of from 0 to 30 wt %.

By an olefin is understood an organic compound containing at least two carbon atoms connected by a double bond. The olefin can be a mono-olefin, having one double bond, or a poly-olefin, having two or more double bonds. Preferably olefins present in the olefinic co-feed are mono-olefins. C4 olefins, also referred to as butenes (1-butene, 2-butene, iso-butene, and/or butadiene), in particular C4 mono-olefins, are preferred components in the olefinic co-feed.

Preferably the olefinic co-feed is at least partially obtained by a recycle stream formed by recycling a suitable fraction of the reaction product comprising C4 olefin. The skilled artisan knows how to obtain such fractions from the olefinic reaction effluent such as by distillation.

In one embodiment at least 70 wt % of the olefinic co-feed, during normal operation, is formed by the recycle stream, preferably at least 90 wt %, more preferably at least 99 wt %. Most preferably the olefinic co-feed is during normal operation formed by the recycle stream, so that the process converts oxygenate feedstock to predominantly light olefins without the need for an external olefins stream. During normal operation means for example in the course of a continuous operation of the process, for at least 70% of the time on stream. The olefinic co-feed may need to be obtained from an external source, such as from a catalytic cracking unit or from a naphtha cracker, during start-up of the process, when the reaction effluent comprises no or insufficient C4+ olefins.

The C4 fraction contains C4 olefin(s), but can also contain a significant amount of other C4 hydrocarbon species, in particular C4 paraffins, because it is difficult to economically separate C4 olefins and paraffins, such as by distillation.

In one embodiment the olefinic co-feed and preferably also the recycle stream comprises C4 olefins and less than 10 wt % of C5+ hydrocarbon species, more preferably at least 50 wt % of C4 olefins, and at least a total of 70 wt % of C4 hydrocarbon species.

The olefinic co-feed and preferably also the recycle stream, can in particular contain at least a total of 90 wt % of C4 hydrocarbon species. In one embodiment, the olefinic co-feed comprises less than 5 wt % of C5+ olefins, preferably less than 2 wt % of C5+ olefins, even more preferably less than 1 wt % of C5+ olefins, and likewise the recycle stream. In another embodiment, the olefinic co-feed, comprises less than 5 wt % of C5+ hydrocarbon species, preferably less than 2 wt % of C5+ hydrocarbon species even more preferably less than 1 wt % of C5+ hydrocarbon species, and likewise the recycle stream.

Thus in certain preferred embodiments, the olefinic portion of the olefinic co-feed, and of the recycle stream, comprises at least 90 wt % of C4 olefins, more preferably at least 99 wt %. Butenes as co-feed have been found to be particularly beneficial for high ethylene selectivity. Therefore one particularly suitable recycle stream consists essentially, i.e. for at least 99 wt %, of 1-butene, 2-butene (cis and trans), isobutene, n-butane, isobutane, butadiene.

In further embodiments the recycle stream can contain a larger fraction of C5 and/or higher olefins. It is for example possible to recycle more than 50% or substantially all of the C5 olefins in the reactor effluent.

In certain embodiments, the recycle stream can also comprise propylene. This may be preferred when a particularly high production of ethylene is desired, so that part or all of the propylene produced is recycled together with C4 olefins.

The preferred molar ratio of oxygenate in the oxygenate feedstock to olefin in the olefinic co-feed depends on the specific oxygenate used and the number of reactive oxygen-bonded alkyl groups therein. Preferably the molar ratio of oxygenate to olefin in the total feed lies in the range of 20:1 to 1:10, more preferably in the range of 15:1 to 1:5.

In a preferred embodiment wherein the oxygenate comprises only one oxygen-bonded methyl group, such as methanol, the molar ratio preferably lies in the range of from 20:1 to 1:5 and more preferably in the range of 15:1 to 1:2.5.

In another preferred embodiment wherein the oxygenate comprises two oxygen-bonded methyl groups, such as for example dimethylether, the molar ratio preferably lies in the range from 10:1 to 1:10.

The process of the present invention can be carried out in a batch, continuous, semi-batch or semi-continuous manner. Preferably the process of the present invention is carried out in a continuous manner.

If the process is carried out in a continuous manner, the process may be started up by using olefins obtained from an external source for the olefinic co-feed, if used. Such olefins may for example be obtained from a steam cracker, a catalytic cracker, alkane dehydrogenation (e.g. propane or butane dehydrogenation). Further, such olefins can be bought from the market.

When a molecular sieve having more-dimensional channels such as ZSM-5 is present in the oxygenate conversion catalyst, even in minority compared to the molecular sieve having one-dimensional 10-membered ring channels, start up is possible without an olefinic co-feed from an external source. ZSM-5 for example is able to convert an oxygenate to an olefin-containing product, so that a recycle can be established.

Typically the oxygenate conversion catalyst deactivates in the course of the process. Conventional catalyst regeneration techniques can be employed, such as burning of coke in a regenerator.

The molecular sieve having one-dimensional 10-membered ring channels used in the process of the present invention can have any shape known to the skilled person to be suitable for this purpose, for it can be present in the form of spray-dried particles, spheres, tablets, rings, extrudates, etc. Extruded catalysts can be applied in various shapes, such as, cylinders and trilobes. Spray-dried particles allowing use in a fluidized bed or riser reactor system are preferred.

Spherical particles are normally obtained by spray drying. Preferably the average particle size is in the range of 1-200 μm, preferably 50-100 μm.

The reactor system used to produce the olefins may be any reactor known to the skilled person and may for example contain a fixed bed, moving bed, fluidized bed, riser reactor and the like. In one embodiment a riser reactor system can be used, in particular a riser reactor system comprising a plurality of serially arranged riser reactors. In another embodiment, a fast fluidized bed reactor can be used.

The reaction to produce the olefinic reaction product can be carried out over a wide range of temperatures and pressures. Suitably, however, the oxygenate feed and olefinic co-feed are contacted with the molecular sieve at a temperature in the range of from 200° C. to 650° C. In a further preferred embodiment the temperature is in the range of from 250° C. to 630° C., more preferably in the range of from 300° C. to 620° C., most preferably in the range of from 450° C. to 600° C. Preferably the reaction to produce the olefinic reaction product is conducted at a temperature of more than 450° C., preferably at a temperature of 460° C. or higher, more preferably at a temperature of 490° C. or higher. At higher temperatures a higher activity and ethylene selectivity is observed. Molecular sieves having one-dimensional 10-membered ring channels can be operated under oxygenate conversion conditions at such high temperatures with acceptable deactivation due to coking, contrary to molecular sieves with smaller pores or channels, such as 8-membered ring channels. Temperatures referred to hereinabove represent reaction temperatures, and it will be understood that a reaction temperature can be an average of temperatures of various feed streams and the catalyst in the reaction zone.

In addition to the oxygenate, and the olefinic co-feed (when present), a diluent may be fed into the reactor system, for example in the range of from 0.01 to 10 kg diluent per kg oxygenate feed, in particular from 0.5 to 5 kg/kg. Any diluent known by the skilled person to be suitable for such purpose can be used. Such diluent can for example be a paraffinic compound or mixture of compounds. Preferably, however, the diluent is an inert gas. The diluent can be argon, nitrogen, and/or steam. Of these, steam is the most preferred diluent. It can be preferred to operate with a minimum amount of diluent, such as less than 500 wt % of diluent based on the total amount of oxygenate feed, in particular less than 200 wt %, more in particular less than 100 wt %. Operation without a diluent is also possible.

The olefinic reaction product or reaction effluent is typically fractionated. The skilled artisan knows how to separate a mixture of hydrocarbons into various fractions, and how to work up fractions further for desired properties and composition for further use. The separations can be carried out by any method known to the skilled person in the art to be suitable for this purpose, for example by vapour-liquid separation (e.g. flashing), distillation, extraction, membrane separation or a combination of such methods. Preferably the separations are carried out by means of distillation. It is within the skill of the artisan to determine the correct conditions in a fractionation column to arrive at such a separation. He may choose the correct conditions based on, inter alia, fractionation temperature, pressure, trays, reflux and reboiler ratios.

At least a light olefinic fraction comprising ethylene and/or propylene and a heavier olefinic fraction comprising C4 olefins are normally obtained. The heavier olefinic fraction preferably contains less than 10 wt % of C5+ hydrocarbon species. Preferably also a water-rich fraction is obtained. Also a lighter fraction comprising methane, carbon monoxide, and/or carbon dioxide can be obtained, as well as one or more heavy fractions comprising C5+ hydrocarbons. Such a heavy fraction, that is not being recycled, can for example be used as gasoline blending component.

In a particular aspect the present invention provides a process for the preparation of an olefinic product, wherein use is made of the sulphur containing catalyst of the present invention, which process comprises the step a) of reacting an oxygenate feedstock and an olefinic co-feed in a reactor in the presence of oxygenate conversion catalyst particles comprising both a molecular sieve having one-dimensional 10-membered ring channels, and a molecular sieve having more-dimensional channels, to prepare an olefinic reaction effluent. Preferably the weight ratio between the one-dimensional molecular sieve and the more-dimensional molecular sieve is in the range of from 1:1 to 100:1. In a preferred embodiment, this process comprises the further steps of b) separating the olefinic reaction effluent into at least a first olefinic fraction and a second olefinic fraction; and c) recycling at least part of the second olefinic fraction obtained in step b) to step a) as olefinic co-feed; and d) recovering at least part of the first olefinic fraction obtained in step b) as olefinic product.

In step b) of this process according to the invention the olefinic reaction effluent of step a) is separated (fractionated). At least a first olefinic fraction and a second olefinic fraction, preferably containing C₄ olefins, are obtained. The first olefinic fraction typically is a light olefinic fraction comprising ethylene, and the second olefinic fraction is typically a heavier olefinic fraction comprising C4 olefins.

Preferably also a water-rich fraction is obtained. Also a lighter fraction comprising contaminants such as methane, carbon monoxide, and/or carbon dioxide can be obtained and withdrawn from the process, as well as one or more heavy fractions comprising C5+ hydrocarbons, including C5+ olefins. Such heavy fraction can for example be used as gasoline blending component. For example, the first olefinic fraction can comprise at least 50 wt %, preferably at least 80 wt %, of C1-C3 species, the recycled part of the second olefinic fraction can comprise at least 50 wt % of C₄ species, a heavier carbonaceous fraction that is withdrawn from the process can comprise at least 50 wt % of C₅₊ species.

In step c) at least part of the second olefinic fraction, preferably containing C₄ olefins, obtained in step b) is recycled to step a) as olefinic co-feed.

Only part of the second olefinic fraction or the complete second olefinic fraction may be recycled to step a).

In the process also a significant amount of propylene is normally produced. The propylene can form part of the light olefinic fraction comprising ethene, and which can suitably be further fractionated into various product components. Propylene can also form part of the heavier olefinic fraction comprising C4 olefins.

The various fractions and streams referred to herein, in particular the recycle stream, can be obtained by fractionating in various stages, and also by blending streams obtained during the fractionation. Typically, an ethylene- and a propylene-rich stream of predetermined purity such as pipeline grade, polymer grade, chemical grade or export quality will be obtained from the process, and also a stream rich in C4 comprising C4 olefins and optionally C4 paraffins, such as an overhead stream from a debutaniser column receiving the bottom stream from a depropanizer column at their inlet. It shall be clear that the heavier olefinic fraction comprising C4 olefins, forming the recycle stream, can be composed from quantities of various fractionation streams. So, for example, some amount of a propylene-rich stream can be blended into a C4 olefin-rich stream. In a particular embodiment at least 90 wt % of the heavier olefinic fraction comprising C4 olefins can be formed by the overhead stream from a debutaniser column receiving the bottom stream from a depropanizer column at their inlet, more in particular at least 99 wt % or substantially all.

Suitably the olefinic reaction effluent comprises less than 10 wt %, preferably less than 5 wt %, more preferably less than 2 wt %, e.g. less than 1 wt %, of C6-C8 aromatics, based on total hydrocarbons. Producing low amounts of aromatics is desired since any production of aromatics consumes oxygenate which is therefore not converted to lower olefins.

The invention also provides an oxygenate conversion catalyst comprising a molecular sieve having n-membered ring channels wherein n is 10 or less, and a sulphur species. n can in particular be 6 or larger, more in particular n can be 8 or 10. In a particular embodiment, n equals 10. The sulphur species is introduced into the catalyst after the synthesis of the molecular sieve, and prior to use in an oxygenate and/or hydrocarbon conversion reaction. At least part of the sulphur species is present in or on the molecular sieve, in particular on the outer surface thereof. Preferably the sulphur species is an inorganic sulphur species, more preferably comprises a SO_{X} species such as at least one of SO₄²⁻ (sulphate) and SO₃⁻ (sulfite). The molecular sieve can comprise a molecular sieve having one-dimensional channels and/or molecular sieve having more-dimensional channels. The invention also provides the use of an oxygenate conversion catalyst as discussed hereinbefore, comprising a molecular sieve having n-membered ring channels wherein n is 10 or less, and a sulphur species, for the conversion of an oxygenate feed, and optionally an olefinic co-feed, to an olefinic product. The oxygenate conversion catalyst is typically the oxygenate conversion catalyst as described herein.

According to a further aspect of the present invention there is provided a process for the manufacture of the oxygenate conversion catalyst, the process comprising:

(a) contacting a catalyst comprising a molecular sieve, wherein the molecular sieves comprises a SAPO-type silicoaluminophosphate, MFI-type aluminosilicate and/or MEL-type aluminosilicate, with a sulphur containing compound; then,

(b) calcining the contacted catalyst;

such that the calcined catalyst comprises a sulphur species at an elemental sulphur loading of at least 0.05 wt % based on total catalyst.

According to an even further aspect of the present invention there is provided an oxygenate conversion catalyst comprising a molecular sieve, wherein the molecular sieves comprises a SAPO-type silicoaluminophosphate, MFI-type aluminosilicate, MEL-type aluminosilicate and/or comprises an aluminosilicate having one-dimensional 10-membered ring channels, and a sulphur species.

The sulphur loading is can be in the range of from 0.05 to 5.0 wt %, preferably of from 0.05 to 2.0 wt % based on total catalyst.

The sulphur loading is can in particular be at least 0.05 wt %, in particular if from 0.05 to 2 wt %, based on molecular sieve in the catalyst.

Preferably the sulphur containing compound is impregnated into the catalyst. During impregnation, a predetermined amount of a solution, such as an aqueous solution, of the sulphur compound is blended with a predetermined quantity of catalyst. After evaporation of the solvent, a controlled amount of the sulphur compound is left on the catalyst.

The method may include combining the molecular sieve with a matrix material as described herein to produce a formulated catalyst. The sulphur containing compound may be contacted with the molecular sieve or catalyst before or after said combination.

The sulphur containing compound may be selected from the group consisting of H₂SO₄, H₂SO₃, H₂SO₇, Na₂SO₄, and (NH₄)₂SO₄. Preferably the sulphur containing compound does not include metal cations.

The catalyst may also comprise phosphorus or a phosphorus species, e.g. such as PO₄³⁻, P—(OCH₃)₃ and/or P₂O₅.

Calcining is herein defined as heating the catalyst to a temperature of above 250° C., preferably above 350° C., for at least 30 minutes, preferably at least 4 hours, optionally in the presence of an inert gas and/or oxygen and/or steam

Typically the sulphur containing compound is converted to the final sulphur species on the catalyst during calcination. In particular it can be oxidised to at least one of SO₄²⁻ and SO₃⁻ following calcination.

Embodiments of the present invention will now be described by way of example only. A series of catalyst samples were prepared comprising 40 wt % zeolite (8 wt % ZSM-5 being a first molecular sieve, and 32 wt % ZSM-23 being a further molecular sieve), 36 wt % kaolin and 24 wt % silica and various amounts of sulphur as detailed in the table below. The samples were then catalytically tested and compared to a sample with a negligible amount of sulphur (<0.01 wt % based on total catalyst).

In the samples ZSM-23 zeolite powder with a silica-to-alumina molar ratio (SAR) of 46, ZSM-5 with a SAR of 280 zeolite powder were used in the ammonium form in the weight ratio 80:20. The powder mix was added to an aqueous solution and subsequently the slurry was milled. Next, kaolin clay and a silica sol were added and the resulting mixture was spray dried wherein the weight-based average particle size was between 70-90 μm. The spray dried catalysts were exchanged by exposure to an ammonium nitrate solution.

Sulphur was deposited on the catalyst by means of impregnation using aqueous solutions acidified by sulphuric acid (H₂SO₄). Different concentrations of the solution were used for depositing different sulphur loadings. After impregnation the catalysts were dried at 120° C. and were calcined at 600° C. for 2 hours. The sulphur loading on the final catalysts was determined by elemental analysis and given in Table 1. The detection limit for S by elemental analysis is below 0.01 wt %. The sulphur content is given as the weight percentage of elemental sulphur on the total weight of the catalyst. The loadings obtained for the calcined catalysts are given in the Table.

To test the samples for catalytic activity and selectivity the respective catalyst powder was pressed into tablets and the tablets were broken into pieces and sieved. Methanol and 1-butene were reacted over the catalysts which were tested to determine their selectivity towards olefins, mainly ethylene and propylene from oxygenates. For the catalytic testing, the sieve fraction of 60-80 mesh was used. Prior to reaction, the catalyst was treated ex-situ in air at 600° C. for 2 hours.

The reaction was performed using a quartz reactor tube of 1.8 mm internal diameter. The catalyst samples were heated in nitrogen to 525° C. and a mixture consisting of 3 vol % 1-butene, 6 vol % methanol, balanced in N₂ was passed over the catalyst at atmospheric pressure (1 bar). The Gas Hourly Space Velocity (GHSV) is determined by the total gas flow over the catalyst weight per unit time (ml.g_{catalyst}⁻¹.h⁻¹). The gas hourly space velocity used in the experiments was 24,000 (ml.g_{catalyst}₋₁.h^{'1}). The effluent from the reactor was analyzed by gas chromatography (GC) to determine the product composition. The composition has been calculated on a weight basis of all hydrocarbons analyzed. The selectivity has been defined by the division of the mass of product by the sum of the masses of all products.

Table 1 shows the results obtained for a series of catalyst samples. Cn refers to hydrocarbon species having n carbon atoms, Cn+ refers to hydrocarbon species having n or more carbon atoms (n being an integer) figures include all; Cn= refers to olefinic hydrocarbon species having n carbon atoms. The index sats refers to saturated carbon species, and tot or totals refer to all respective hydrocarbon species.

TABLE 1

| | Untreated | | | | | | | |
|---|---|---|---|---|---|---|---|---|
| | (comparative) | | H₂SO₄ | | H₂SO₄ | | H₂SO₄ | |
| | S-content (wt % of total caralyst) | | | | | | | |
| | <0.01 | | 0.1 | | 0.3 | | 0.4 | |
| Time (h) | 2 | 10 | 2 | 10 | 2 | 10 | 2 | 10 |
| DME/methanol | 100 | 100 | 100 | 100 | 100 | 100 | 100 | 100 |
| conversion (%) | | | | | | | | |
| C2-C5 wt % | 94.0 | 91.3 | 94.9 | 93.4 | 93.7 | 92.4 | 94.3 | 92.8 |
| C6+ wt % | 5.2 | 7.7 | 4.3 | 6.0 | 4.9 | 6.7 | 4.7 | 6.5 |
| C7-C8 aromatics wt % | 0.9 | 1.0 | 0.8 | 0.6 | 0.9 | 0.5 | 0.5 | 0.4 |
| C4 sats/C4total wt/wt | 7.3 | 9.9 | 5.9 | 6.6 | 5.3 | 5.7 | 5.3 | 5.6 |

C2-C5 products include paraffins and olefins, and comprise at least 90 wt % of olefins. C6+ excludes toluene, xylenes, ethylbenzene

As can be observed from the Table, with increasing sulphur content, the make of unwanted by-products C7-C8 is gradually reduced. The preferred product, C2-C5, does not decrease with increasing amount of sulphur and indicates that catalyst activity is sustained. Another beneficial effect is that the ratio C4 saturates/C4 total is also decreased after deposition of sulphur, and C4 saturates are unwanted by-products particularly when a C4 stream is to be recycled to the oxygenate conversion reaction.

Thus the above results show an improved selectivity of embodiments of the present invention comprising a

1. A process for the preparation of an olefinic product in the presence of an oxygenate conversion catalyst, the catalyst comprising a molecular sieve, wherein the molecular sieves comprises a SAPO-type silicoaluminophosphate, MFI-type aluminosilicate, MEL-type aluminosilicate and/or an aluminosilicate having one-dimensional 10-membered ring channels, and a sulphur species at an elemental sulphur loading of at least 0.05 wt % based on total catalyst,
the process comprising reacting an oxygenate feedstock under oxygenate conversion conditions to produce an olefinic reaction product.

2. A process as claimed in claim 1, wherein the molecular sieve comprises an aluminosilicate having one-dimensional 10-membered ring channels.

3. A process as claimed in claim 1, wherein the molecular sieve comprises a first molecular sieve having more-dimensional channels and a further molecular sieve having one-dimensional 10-membered ring channels.

4. A process as claimed in claim 3, wherein the catalyst has catalyst particles, and wherein individual catalyst particles comprise both the first molecular sieve and the further molecular sieve.

5. A process as claimed in claim 1, wherein the sulphur species comprises sulfite and/or sulphate.

6. A process as claimed in claim 1, wherein the catalyst has an elemental sulphur loading of from 0.05 to 2 wt %.

7. A process as claimed in claim 1, wherein the catalyst further comprises phosphorus or a phosphorus species loaded on the catalyst after catalyst formulation.

8. A process as claimed in claim 1, wherein the oxygenate feedstock is reacted to produce the olefinic reaction product in the presence of an olefinic co-feed.

9. A process for the manufacture of an oxygenate conversion catalyst, the process comprising:
(a) contacting a catalyst comprising a molecular sieve, wherein the molecular sieves comprises a SAPO-type silicoaluminophosphate, MFI-type aluminosilicate, MEL-type aluminosilicate and/or comprises an aluminosilicate having one-dimensional 10-membered ring channels, with a sulphur containing compound; then,
(b) calcining the contacted catalyst;
such that the calcined catalyst comprises a sulphur species at an elemental sulphur loading of at least 0.05 wt % based on total catalyst.

10. A process as claimed in claim 9, wherein the sulphur containing compound is impregnated into the catalyst.

11. A process as claimed in claim 9, wherein the sulphur containing compound is selected from the group consisting of H₂SO₄, H₂SO₃, H₂SO₇, Na₂SO₄, and (NH₄)₂SO₄.

12. A process as claimed in claim 9, wherein the catalyst also comprises phosphorus or a phosphorus containing compound, preferably loaded on the catalyst after catalyst formulation.

13. An oxygenate conversion catalyst comprising a molecular sieve, wherein the molecular sieves comprises a SAPO-type silicoaluminophosphate, MFI-type aluminosilicate, MEL-type aluminosilicate and/or comprises an aluminosilicate having one-dimensional 10-membered ring channels, and a sulphur species.
